(19) United States
(12) Patent Application Publication
(10) Pub. No.: US 2025/0288293 A1
(43) Pub. Date: Sep. 18, 2025

(54) CORDED NON-INVASIVE WOUND CLOSURE DEVICE WITH RIGID SLIDERS AS REDUNDANT SUPPORTS

(71) Applicant: Jamil Mogul, Santa Clara, CA (US)

(72) Inventor: Jamil Mogul, Santa Clara, CA (US)

(21) Appl. No.: 19/226,005

(22) Filed: Jun. 2, 2025

Related U.S. Application Data
(63) Continuation-in-part of application No. 18/088,401, filed on Dec. 23, 2022.

(51) Int. Cl. A61B 17/08

(52) U.S. Cl. CPC A61B 17/085; A61B 2017/088

(57) ABSTRACT

A non-invasive wound closure device that does not use needles, sutures, staples or tissue-adhesives. The device reapproximates the everted edges of a wound of a patient, and has two side strips each containing a customized bandage, that are adhered to either side of the wound that is closed by mating and tying the two tethered side strips via the cord and the rigid slider(s) and the locking mechanism. The device can also be reopened by untying the cord and removing the slider(s), and can be reclosed by repeating the closure process. Each side strip has cross holes, through which the cord tethers, joins and ties the side strips, and has an integral adhesive-bandage layer on the bottom to adhere to the patient's skin. The device may be made in various lengths and be used singly or in segments for straight wounds, or using multiple sliders for curved or dynamic wounds.

### CROSS REFERENCE TO RELATED APPLICATIONS

This application is a Continuation-in-Part of application Ser. No. 18/088,401, “Non-Invasive Wound Closure Device”, filed on Dec. 23, 2022, currently pending.

### FIELD OF THE INVENTION

The present invention relates to an apparatus that reapproximates the everted edges of a wound or surgical cut without sutures or staples, or any invasive surgical tool or needle. Often no anesthetics are required, which allows the practitioner to inspect, reinspect, reoperate on if necessary, and reclose the wound or cut without any invasive surgical tool or needle.

### SUMMARY

Sutures used for closing a wound or surgical cut are made of synthetic or natural materials. Certain suture materials may cause an allergic reaction to a patient. Some sutures are absorbable while others need to be removed once sufficient wound healing has occurred. Wound infection can occur in skin surgeries, depending upon the type of procedure, type and location of the wound or cut, and patient factors. Complications after a given procedure include increasing redness, swelling, fever, pain around the wound or cut, or pus or discharge from the wound or cut. Additionally, sutures pose a risk of needle-stick injury to the patient and also to the practitioner.

Another complication of suturing is wound or cut dehiscence, which can occur when a wound or cut heals properly and stitches are removed. The sutures around the edges of the wound or cut stay intact while a granulation tissue or granuloma starts forming. It is, among other factors, often due to utilization of an inappropriate suture knot, tying the suture knots too loosely, employment of an improper suture material, over-tightening or misplacement of the suture, and/or an incorrect suture technique. A clean reopened wound or cut can be re-stitched but an infected wound or cut is often left to heal. Absorbable sutures can sometime extrude through the skin as they dissolve. Late complications include scars or hypertrophic scarring and keloid formations, which may be due to either improper suturing with excess tension or lack of eversion of the edges. Other complications include stitch marks and wound necrosis.

Stapling, which is much faster than suturing, may also cause scars in the skin similar to sutures. But the scars resulting from staples are often more pronounced. Patient acceptance and comfort and wound infection and dehiscence are similar with wounds or cuts closed with staples and sutured wounds or cuts. However, removal of staples can be somewhat more uncomfortable than removal of sutures. Also, cosmesis suffers, especially if the staples are left in too long.

Wounds or surgical cuts closed with tissue adhesives have been found to have rates of healing and complications that are equivalent to those of staples-closed wounds or cuts and sutured wounds or cuts. Additionally, if an excessive amount of adhesive is applied too quickly, the patient can experience and sustain a local burn from the heat of polymerization. And if the adhesive is washed or soaked, it will peel off in a few days, before the wound is healed. Also, if the adhesive contacts the clinician's gloved fingers, the glove may adhere to the patient's skin. There is a slightly higher risk for wound or cut dehiscence in wound closures with adhesives than with sutures. Tissue adhesives cannot be used for closing wounds or cuts in a high-tension skin area. Similarly, they cannot be used near the eyes, over or near joints, on moist or mucosal areas, or on wounds or cuts under a highly static or dynamic skin tension.

Adhesive tapes or strips used for closing wounds or surgical cuts often lead to inferior cosmetic results, and premature tape separation may often occur. Other complications include skin blistering if no cross-strips are used or the tape is stretched too tightly across the wound, and hematoma may occur if hemostasis is inadequate. Also, tape may loosen prematurely over shaved areas as hair grows back.

As they are used across a wound, adhesive strips with a hinge, pull tabs and ratchet locks, which are functionally and operationally similar to the adhesive tapes or strips that are commonly used for wound closures, may have similar complications. Also, adhesive strips with a hinge, pull tabs and ratchet locks do not have provisions for reinspection of, or reoperation on and reclosure of the wound, as the tabs are cut off after closing the wound. Even if the tabs are not cut off, the ratchet locks are one-way and do not allow the sides of the strip placed across the wound to be moved back or brought to the strip's initial state.

Similarly, the mesh and hooks strips cannot be disengaged easily for reinspection of or reoperation on the wound without distorting or breaking the hooks or receptacle mesh holes. Hence the strips cannot be effectively re-engaged to re-close the wound.

To address the issues and complications mentioned above, a non-invasive wound closure device is disclosed that reapproximates the everted edges of a wound or cut often without any anesthetics faster, more simply and safely, and less painfully than staples, sutures, tissue adhesives or adhesive tapes, or the abovementioned non-invasive strips with pull tabs/hinge/ratchet locks, or mesh and hooks strips.

In a preferred embodiment of the present disclosure, the wound closure device includes two side strips. Each side strip is, on a bottom side thereof, attached to a custom bandage. Each side strip includes on a top side thereof an offset protrusion along its entire length. The protrusions include alignment holes, and form a wedge tongue when the protrusions are joined together. A longitudinal slider strip has a transversely blind slot to form a wedge groove that matches the conformation of the protrusions.

The custom bandage is a dimensionally modified version of a typical adhesive bandage with a peel-away backing. The modification is made to lengthen the fabric absorbent to the length of the side strip and to offset the fabric absorbent to one side.

To reapproximate a wound that has been medicinally prepped, once the backing is peeled off, a first side strip is placed on and adhered to one side of the wound. The user should ensure that the protrusion is parallel to and adjacent to the wound's edge. For the other side of the wound, the procedure is repeated with a second side strip while making sure that the protrusion of the second side strip faces the protrusion of the first side strip.

Both placed side strips are then pressed down by the protrusions and brought toward each other to evert the edges of the wound. The side strips are mated by aligning the protrusion holes and joining the protrusions together. Then the slider strip is installed by inserting the joined protrusions into the slider strip slot, and by sliding the slider strip completely into the slider strip slot. This keeps the two side strips mated, resulting in wound closure.

To reinspect and/or reoperate on the wound, the slider strip is slid out, which will separate the mated side strips and thereby provide a means to access the wound. To reclose the wound, both placed side strips are again mated.

Along the entire length of the offset protrusion of the side strip, there is a longitudinal groove on the longitudinal, angled side of the protrusion. The groove centers and intersects a plurality of alignment holes. The plurality of alignment holes and the groove may be used to receive a sealing cord.

The slider strip, or slider, is a rigid element and has a through cross hole on the sides. The sliders can be of any length desired by the user, and can be used singly or in groups of multiple elements. The through cross hole on the sides of the rigid slider serves as a part of a locking mechanism for the rigid slider when it is installed.

Along the length of the offset protrusion of the side strip and above the alignment holes is a longitudinal array of a plurality of soft, spherical protuberances on the longitudinal, angled side of the protrusion. These protuberances diametrically match the cross hole and serve as another part of the locking mechanism for the rigid slider.

For curved and/or dynamic wounds, the cord is used as a main means of joining the protrusions of the side strip together. The two side strips that are juxtaposed with their protrusions facing each other are tethered together by threading the cord whose ends are inserted from one side of each side strip alternately into all the protrusion holes. The ends of the threaded cord exit alternately from the last protrusion holes and extend out at the other side of each side strip.

To reapproximate a wound that has been medicinally prepped, once the backing of the side strip is peeled off, the side strip is placed on and adhered to one side of the wound while making sure that the protrusion is parallel and adjacent to the wound's edge. For the other side of the wound, the same is done using the other side strip while making sure that its protrusion faces the protrusion of the other placed side strip.

Now, both placed side strips are pressed down by the protrusions and brought toward each other to evert the edges of the wound and mated by joining the protrusions together while aligning the protrusion holes and by pulling up all the way and tying the extended ends of the threaded cord with an untieable knot, which keeps the two side strips mated, resulting in a secure wound closure.

To reinspect and/or reoperate on the wound, the knot of the cord is untied, which will separate the mated side strips and thereby provide an open access to the wound. And to reclose the wound, both placed side strips are re-mated as mentioned above.

As redundant supports, in case of high or excessive dynamics of the wound, which may cause the cord's knot to get accidently loosened or untied causing the joined protrusions of the side strips to be separated, short rigid sliders are installed onto the joined protrusions and soft-locked in place via the built-in locking mechanism that as alluded above, is comprised of the cross hole on the sides of each rigid slider and the soft, spherical protuberances on the longitudinal, angled side of the protrusion of the side strip.

To reinspect and/or reoperate on the wound that is highly or excessively dynamic, the rigid sliders are removed and the knot of the cord is untied, which will separate the joined side strips and thereby provide an open access to the wound. And to reclose the wound, both placed side strips are re-joined as mentioned above.

An important advantage of the present device is that it is applicable to all types of curved and/or dynamic wounds and due to the use of the cord as a joining means of the side strips, the device has a low profile, which allows the device to be used in space-constrained places on the human body while also allowing the closed wounds or incisions to heal properly.

Another important advantage of the present invention is that thanks to the added rigid sliders, it provides, in case of accidental loosening or untying of the knot of the cord, a redundant support for assuring enhanced security of the closed wounds or incisions that are in the highly or excessively dynamic areas of the human body.

Yet another important advantage of the present invention is that thanks to the built-in locking mechanism, it prevents the rigid sliders from slipping off in case of an accidental hit on it due to a physical activity, while at the same time providing a secure means for the closed wounds or incisions to heal uninterruptedly.

These and other advantages of the present invention will become apparent to those skilled in the art after reading the following detailed disclosure of the embodiment of the present invention.

### BRIEF DESCRIPTION OF THE DRAWINGS

FIG. 1A and FIG. 1B show isometric views of the two side strips each attached to the custom bandage.

FIG. 2 shows an isometric cut-away view of a body part with a linear open wound.

FIG. 3A shows an isometric view of the placement of the side strip on either side of the wound on the body part.

FIG. 3B shows an enlarged isometric cut-away view of the details around the area at the proximal end of the open wound.

FIG. 3C shows an isometric view of the placed side strips that are mated.

FIG. 3D shows an enlarged isometric cut-away view of the details around the area at the proximal end of the closed wound due to mating of the side strips.

FIG. 3E shows an isometric view of the two mated side strips that are captivated and securely locked in place by the installed slider strip.

FIG. 3F shows an enlarged isometric cut-away view of the details around the area at the proximal end of the closed wound and the mated side strips and the installed slider strip.

FIG. 4A and FIG. 4B show isometric views at two different rotational angles of the slider strip.

FIG. 4C shows an enlarged isometric cut-away view of the details around one end of the slider strip.

FIG. 5A and FIG. 5C show isometric views at two different rotational angles of the side strip.

FIG. 5B and FIG. 5D show, respectively, enlarged isometric cut-away views of the details around a corner of the side strip.

FIG. 6A and FIG. 6B show isometric views at two different rotational angles of the custom bandage.

FIG. 6C and FIG. 6D show, respectively, enlarged isometric cut-away views of the details around a corner of the custom bandage.

FIG. 7A and FIG. 7B show isometric views of the two side strips each attached to the custom bandage.

FIG. 7C shows an isometric view of the tethered side strips.

FIG. 8A shows an isometric view of the placement of the side strip on either side of the wound on the body part.

FIG. 8B shows an enlarged isometric cutaway view of the details around the area at the proximal end of the open wound.

FIG. 8C shows an isometric view of the placed side strips that are mated

FIG. 8D shows an enlarged isometric cutaway view of the details around the area at the proximal end of the closed wound due to the mating of the side strips.

FIG. 8E shows an enlarged isometric cutaway view of the details around the area at the distal end of the closed wound and the mated side strips and the knotted ends of the threaded cord.

FIG. 9 shows an isometric view of the cord.

FIG. 10A shows isometric views of multiple rigid sliders.

FIG. 10B shows an enlarged isometric cutaway view of the details of the rigid slider.

FIG. 11A shows an isometric view of the two mated side strips that are redundantly captured and securely locked in place by the installed rigid sliders.

FIG. 11B shows an enlarged isometric cutaway view of the details around the area at the proximal end of the closed wound and the mated side strips and the installed rigid sliders.

FIGS. 12A and 12C show isometric views at two different rotational angles of the side strip.

FIGS. 12B and 12D show, respectively, enlarged isometric cutaway views of the details around a corner of the side strip.

### DETAILED DESCRIPTION

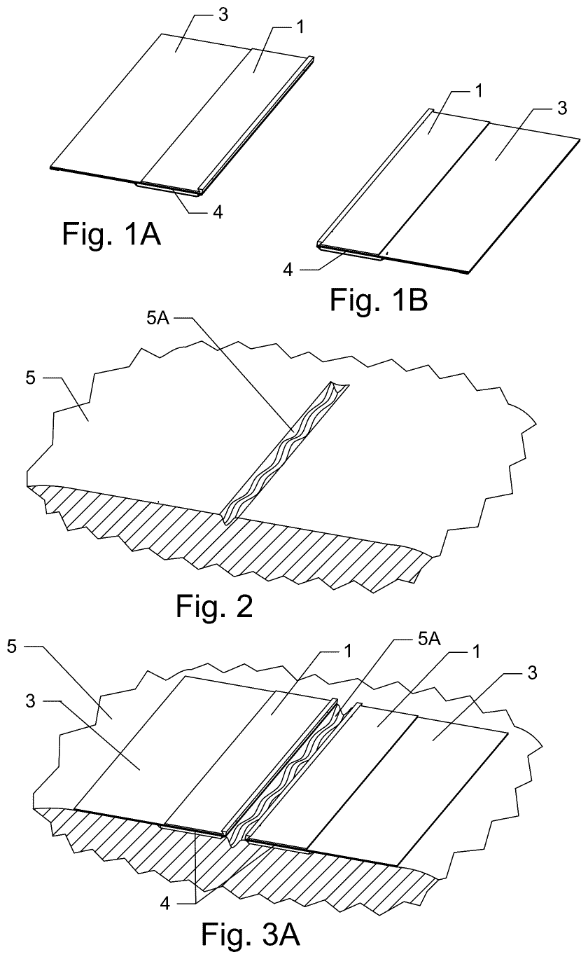

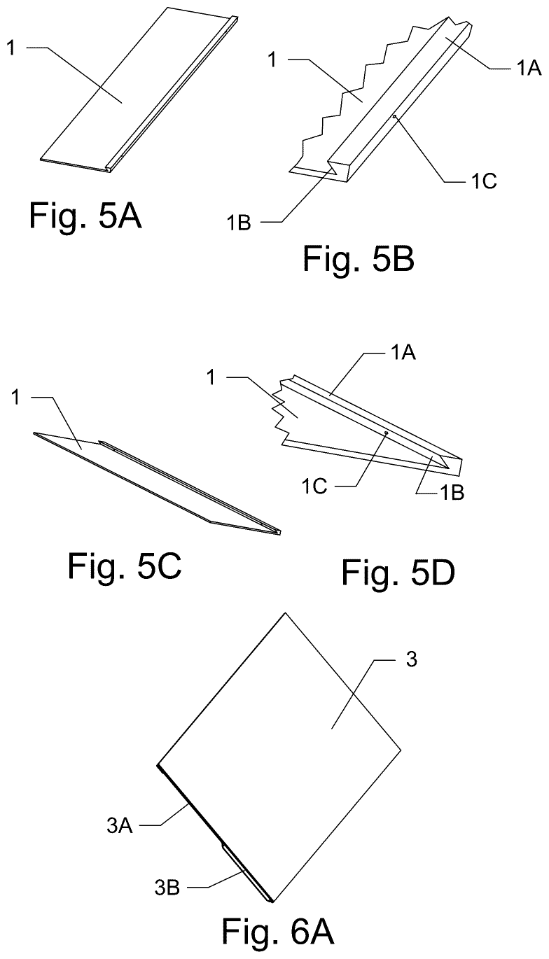

Referring first to the isometric views in FIG. 1A, FIG. 1B, FIGS. 4A, FIG. 4B, FIG. 4C, FIG. 5A, FIG. 5B, FIG. 5C, and FIG. 5D illustrating a preferred embodiment of the non-invasive wound closure device in accordance with the present disclosure, the wound closure device consists of at least two side strips 1, and a bandage 3. The bandage 3 may be chosen in any size or shape by the user. Each side strip 1 on a bottom planar surface has an attachment layer 4. Any mechanical, thermal, electrical, chemical, ultrasonic, photonic, adhesive, or any other bonding or forming means can be the attachment layer 4 affixed to the top non-adhering planar surface of the custom bandage 3.

Each side strip 3 includes an offset protrusion 1A that extends longitudinally along the side strip 1. The protrusion 1A has at least two side holes 1C and an inwardly angled side wall 1B in order for the protrusion 1A to form a wedge tongue when the protrusion 1A of a first strip 1 is aligned with the protrusion 1A of a second side strip 1 by matching manually or via a tool that aligns the holes 1C when the protrusions 1A of the two side strips 1 are brought together.

A sliding fastener called a slider strip 2, which is rigid, has an end-to-end open, transversely blind slot 2A whose side walls are also inwardly angled to match the inwardly angled side wall 1B of the protrusion 1A of the side strip 1 to form a wedge groove.

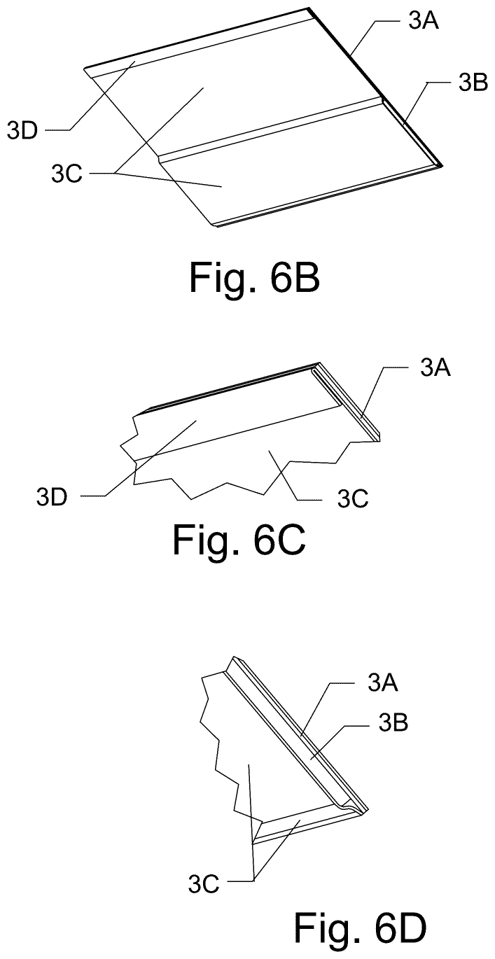

Referring now to FIGS. 6A-6D, which show isometric views of the custom bandage 3, which has an adhesive layer 3A, a fabric absorbent layer 3B, and a peel-away backing 3C. The length of the fabric absorbent layer 3B is generally cut to the length of the side strip 1. The fabric absorbent layer 3B is positioned at one side of the side strip 1 so that the offset fabric absorbent layer 3B is longitudinally continuous from a first end to a second end of the custom bandage 3.

For easier removal, the peel-away backing 3C has a folded end 3D.

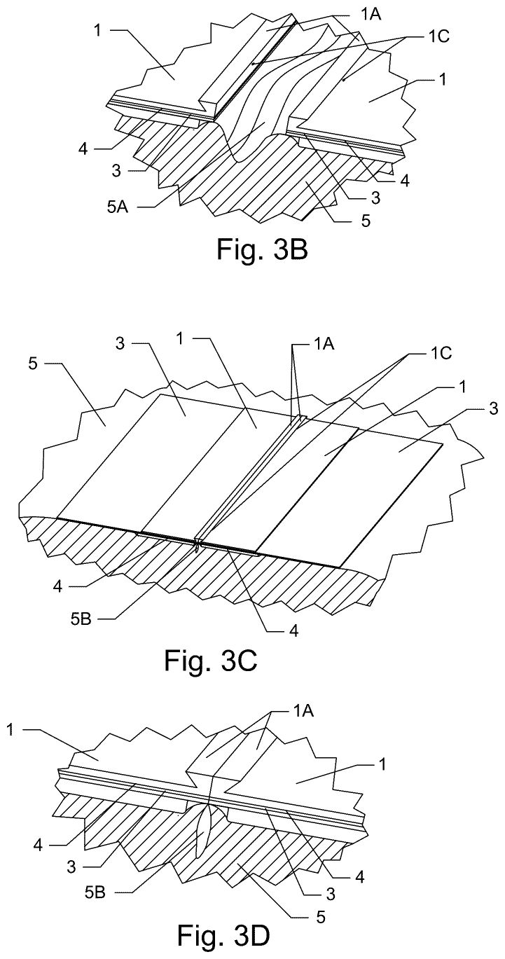

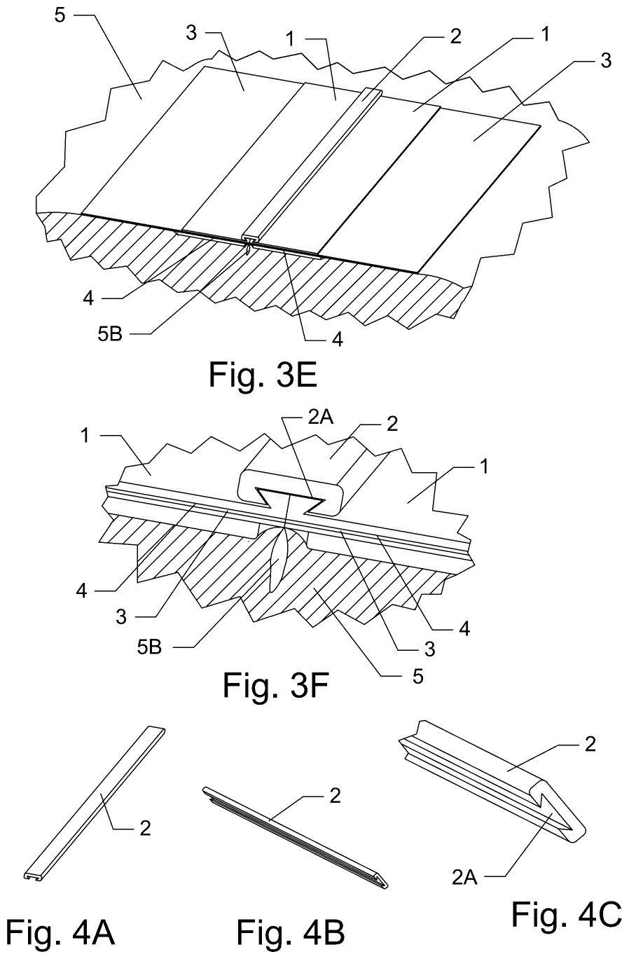

Referring to FIG. 2, FIG. 3A, and FIG. 3B, which show isometric views of a body part of a patient, a body part 5 has an essentially linear open wound 5A. To reapproximate the open wound 5A that has been cleaned and medicinally prepped, the backing 3C of the custom bandage 3 that is integral to the side strip 1 is peeled off from the folded end 3D and then from the side strip 1. The custom bandage 3, is now without the backing 3C so that the adhesive layer 3A is exposed, and is placed on one side of the open wound 5A. The bandage 3 is positioned so that the protrusion 1A of the side strip 1 is parallel and adjacent to a first edge of the open wound 5A. The bandage 3 of the side strip 1 is adhered to the skin of the body part 5 by pressing down on the side strip 1 with a swiping finger or thumb. For a second side of the open wound 5A, the same procedure is followed using the second side strip 1 with the bandage 3 while making sure that the protrusion 1A of the side strip 1 with the bandage 3 faces the protrusion 1A of the first side strip 1 that had been positioned previously.

Referring now to FIG. 3C, FIG. 3D, FIG. 3E, and FIG. 3F, which show isometric views, with a thumb and a finger, the protrusion 1A of the first placed side strip 1 with the custom bandage 3 and the protrusion 1A of the second placed side strip 1 with the custom bandage 3 are pressed down and brought toward each other in order to evert the edges of the open wound 5A and join the two placed side strips 1. The two side strips 1, each with a bandage 3, are mated by aligning and joining the protrusions 1A together manually, or by temporarily inserting an appropriate pin into the alignment holes 1C of the protrusions 1A. While holding the aligned protrusions 1A together, the slider strip 2 is installed by inserting the aligned and joined protrusions 1A, which together form the wedge tongue, into the slot 2A of the slider strip 2, the slot acting as the wedge groove. The slider strip 2 is positioned on the aligned and joined protrusions 1A so as to fully secure the joined protrusions 1A and to securely lock in place the mated side strips 1, each with a custom bandage 3, thereby closing the wound 5B of the body part 5.

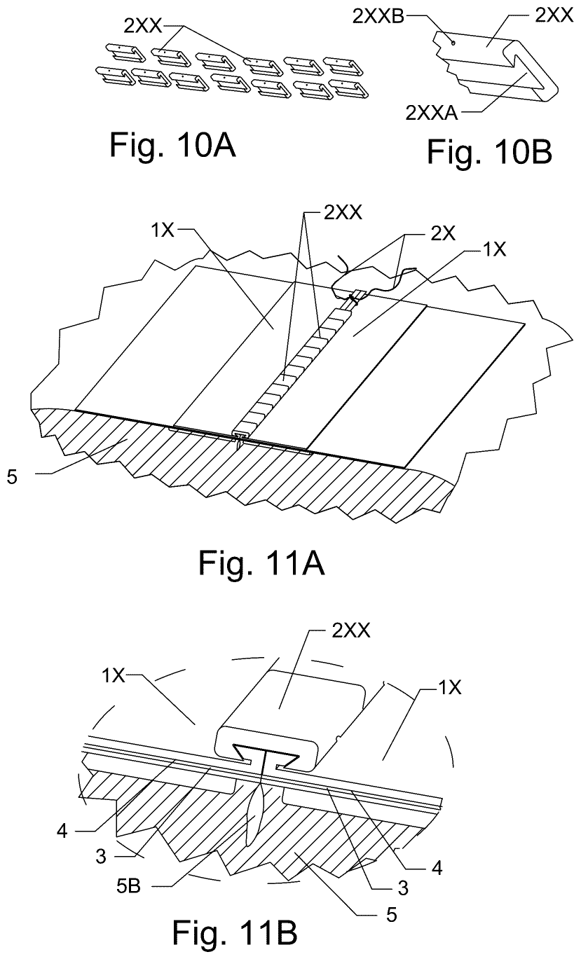

FIGS. 10A and 10B illustrate the way in which curved wounds or incisions can be handled with the device. FIG. 10A shows multiple smaller slider strips or smaller sliders 2XX. The smaller slider 2XX is shown in further detail in FIG. 10B. As is illustrated in FIG. 10A, the rigid slider strip 2 can be constructed as multiple smaller sliders 2XX. In that way, the smaller sliders 2XX, although rigid elements, can approximate a curved device by using multiple units of smaller rigid slider 2XX to form an arced slider strip 2 that follows the pattern of the curved wound or incision.

Later, to reinspect the closed wound 5B and/or to operate again via the reopened wound 5A, the slider strip 2 is slid out, which allows the mated side strips 1 to separate, which provides access to the closed wound 5B, or the reopened wound 5A. To reclose the closed wound 5B or the reopened wound 5A, the two side strips 1 are re-mated as described above.

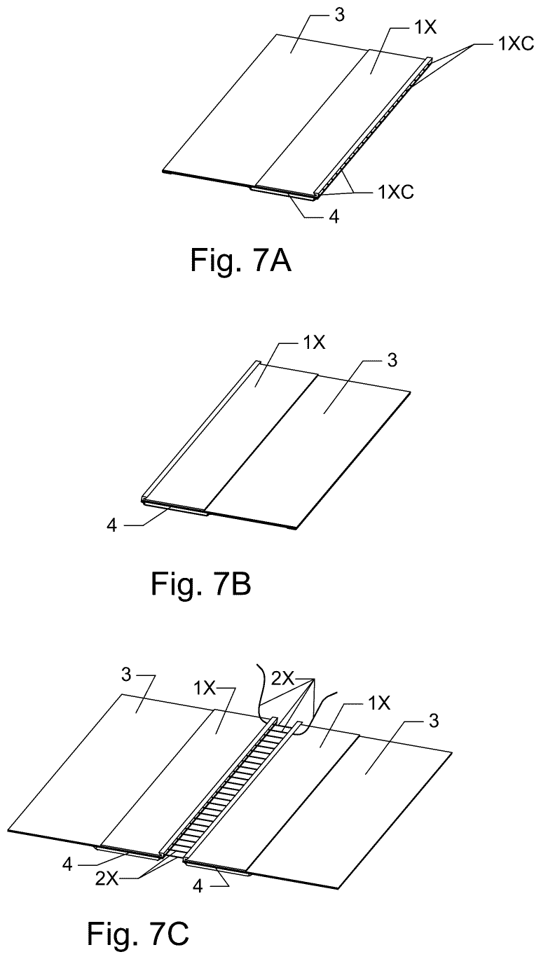

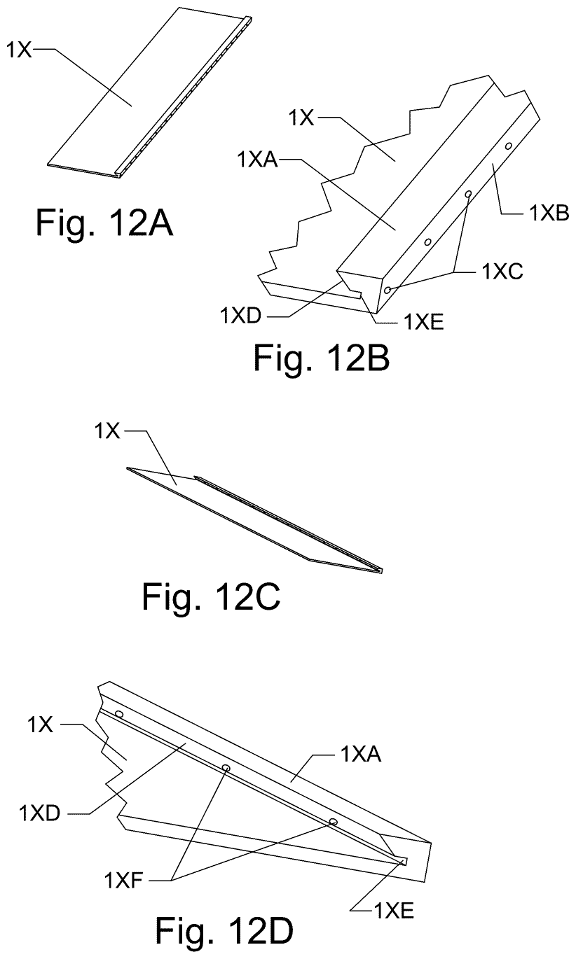

Referring to FIG. 7A, FIG. 7B, FIG. 7C, FIG. 9, FIG. 12A, FIG. 12B, FIG. 12C, and FIG. 12D, which are isometric views, the wound closure device consists of at least two side strips 1X, each with a custom bandage 3 attached to the side strip 1X. The side strip 1X is connected on one surface to the bandage 3. The side strip 1X has on a second surface a longitudinal offset protrusion 1XA. The protrusion 1XA has a perpendicular side 1XB that includes a plurality of cross holes 1XC in a longitudinal array. The protrusion 1XA has an inwardly angled side 1XD so that the side strip 1X can be easily lifted and brought together with the protrusion 1XA of a second side strip 1X. This configuration also allows the inwardly angled side 1XD to function as a wedge tongue.

Also, along the length of the offset protrusion 1XA of the side strip 1X, there is a longitudinal groove 1XE on the inwardly angled side 1XD of the protrusion 1XA. The groove 1XE centers and intersects the holes 1XC that are used for installing a cord 2X. The cord 2X has rigid ends 2XA for ease of handling and threading the cord 2X into the holes 1XC. The groove 1XE of the protrusion 1XA is used for receiving the threaded cord 2X in a position flush with the surface of the inwardly angled side 1XD of the protrusion 1XA.

The first side strip 1X is tethered to the second side strip 1X by the cord 2X whose rigid ends 2XA are inserted from first ends of each side strip 1X alternately into the holes 1XC. The rigid ends 2XA of the cord 2X exit alternately from and extend out of the ending holes 1XC at the second ends of the two side strips 1X that are tethered together.

Referring to FIG. 6A, FIG. 6B, FIG. 6C, and FIG. 6D, the custom bandage 3 is a modified version of a typical bandage that is normally used for covering wounds or skin cuts and that has an adhesive layer 3A, a piece of a fabric absorbent 3B, and a peel-away backing 3C. The modification is made to lengthen the fabric absorbent 3B to the length of the side strip 1X and to offset the fabric absorbent 3B to one side so that the offset fabric absorbent 3B is longitudinally continuous from one end to the other end of the custom bandage 3. For ease of removal, the peel-away backing 3C has a folded end 3D.

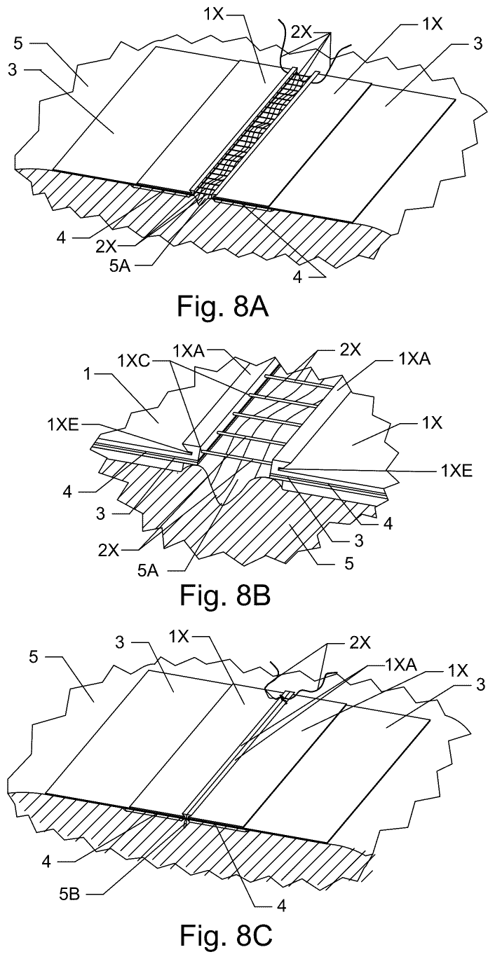

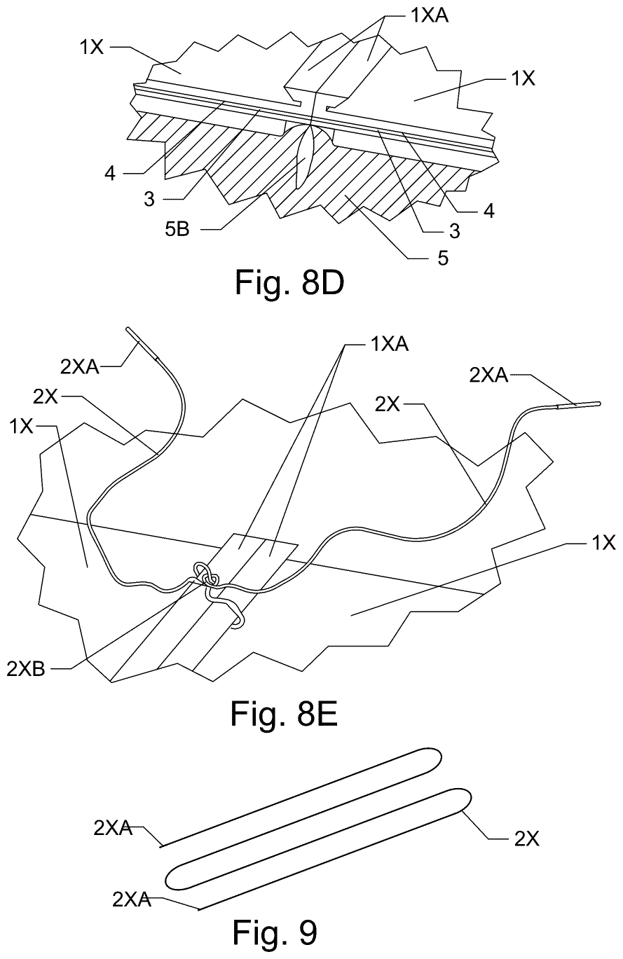

Referring to FIG. 2, FIG. 8A, and FIG. 8B, a body part 5 has an open wound 5A. To reapproximate the open wound 5A that has been cleaned and medicinally prepped, the backing 3C of the custom bandage 3 that is integral to the tethered side strip 1X is peeled off from the folded end 3D. The bandage 3 of the tethered side strip 1X that is now without the backing 3C is placed on one side of the open wound 5A while making sure that the protrusion 1XA of the tethered side strip 1X is parallel and adjacent to the edge of the open wound 5A. The bandage 3 is properly adhered to the skin of the body part 5 by pressing down on the tethered side strip 1X with a swiping finger or thumb. The same procedure is repeated for the other side of the open wound 5A.

Referring to FIG. 8C, FIG. 8D, and FIG. 8E, now, with a thumb and a finger, the protrusion 1XA of the placed tethered side strip 1X with the custom bandage 3 and the protrusion 1XA of the other placed tethered side strip 1X with the custom bandage 3 are pressed down and brought toward each other in order to evert the edges of the open wound 5A and join the two placed tethered side strips 1X, each with the custom bandage 3. The two placed tethered side strips 1X are mated by aligning and joining the protrusions 1XA manually. Then, while holding the aligned protrusions 1XA together and aligning the holes 1XC and by pulling up and tying the extended rigid ends 2XA of the threaded cord 2X into an untieable knot 2XB, the joined protrusions 1XA are fixed in position, and the mated tethered side strips 1X are secured, thereby resulting in closure of the wound 5B of the body part 5.

To reinspect the closed wound 5B and/or to reoperate on the reopened wound 5A, the knot 2XB of the threaded cord 2X is untied, which separates the mated tethered side strips 1X, thereby providing access to the wound. To again close the wound, both tethered side strips 1X, are mated as described above.

Now referring to FIG. 10A, FIG. 10B, FIG. 11A and FIG. 11B, and again referring to FIG. 12A, FIG. 12B, FIG. 12C, and FIG. 12D, there is a plurality of smaller rigid sliders 2XX, each of which has an end-to-end open, transversely blind slot 2XXA whose side walls are also inwardly angled so as to match the inwardly angled side 1XD of the protrusion 1XA of the side strip 1X and to form a wedge groove. For redundant support, in case of high or excessive dynamics of the wound, which may cause the knot 2XB of the cord 2X to get accidently loosened or untied causing the joined tethered side strips 1X to be separated, the smaller rigid sliders 2XX are installed by sliding them onto the joined protrusions 1XA one by one. The wedge tongue of the protrusions 1XA are received in the slot 2XXA of each of the smaller rigid sliders 2XX.

Typically, the smaller rigid sliders 2XX will be in contact with each other after they are installed. There is a through cross hole 2XXB on the sides of each of the smaller rigid sliders 2XX. Along the length of the offset protrusion 1XA of the side strip 1X and above the holes 1XC, there is a longitudinal array of a plurality of soft, spherical protuberances 1XF on the longitudinal, inwardly angled side 1XD of the protrusion 1XA. The protuberances 1XF diametrically match the cross hole 2XXB of the smaller rigid sliders 2XX. The cross hole 2XXB of each of the smaller rigid sliders 2XX and the protuberances 1XF of the protrusion 1XA form a built-in locking mechanism. While they are being installed, the smaller rigid sliders 2XX are soft-locked in place via the built-in locking mechanism.

To reinspect and/or reoperate on the wound, the closed wound 5B is reopened. To do so, the smaller rigid sliders 2XX are removed one by one and the knot 2XB of the cord 2X is untied, which will separate the mated tethered side strips 1X and thereby provide access to the wound. To reclose the reopened wound 5A, the tethered side strips 1X are re-mated as described above.

The description of the present disclosure has been presented for purposes of illustration and description, but is not intended to be exhaustive or limited to the present disclosure in the form disclosed. Many modifications and variations will be apparent to those of ordinary skill in the art without departing from the scope and spirit of the present disclosure. Exemplary embodiments were chosen and described in order to best explain the principles of the present disclosure and its practical application, and to enable others of ordinary skill in the art to understand the present disclosure for various embodiments with various modifications as are suited to the particular use contemplated.

While this technology is susceptible of embodiment in many different forms, there is shown in the drawings and will herein be described in detail several specific embodiments with the understanding that the present disclosure is to be considered as an exemplification of the principles of the technology and is not intended to limit the technology to the embodiments illustrated.

The terminology used herein is for the purpose of describing particular embodiments only and is not intended to be limiting of the technology. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that like or analogous elements and/or components, referred to herein, may be identified throughout the drawings with like reference characters. It will be further understood that several of the figures are merely schematic representations of the present disclosure. As such, some of the components may have been distorted from their actual scale for pictorial clarity.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments.

Furthermore, depending on the context of discussion herein, a singular term may include its plural forms and a plural term may include its singular form. Similarly, a hyphenated term (e.g., “on-demand”) may be occasionally interchangeably used with its non-hyphenated version (e.g., “on-demand”), a capitalized entry (e.g., “Software”) may be interchangeably used with its non-capitalized version (e.g., “software”), a plural term may be indicated with or without an apostrophe (e.g., PE's or PEs), and an italicized term (e.g., “N+1”) may be interchangeably used with its non-italicized version (e.g., “N+1”). Such occasional interchangeable uses shall not be considered inconsistent with each other.

It is noted at the outset that the terms “coupled,” “connected”, “connecting,” “electrically connected,” etc., are used interchangeably herein to generally refer to the condition of being electrically/electronically or optically connected. Similarly, a first entity is considered to be in “communication” with a second entity (or entities) when the first entity electrically sends and/or receives (whether through wireline or wireless means) information signals (whether containing data information or non-data/control information) to the second entity regardless of the type (analog or digital) of those signals. It is further noted that various Figures (including component diagrams) shown and discussed herein are for illustrative purposes only, and are not drawn to scale.

While specific embodiments of, and examples for, the system are described above for illustrative purposes, various equivalent modifications are possible within the scope of the system, as those skilled in the relevant art will recognize. For example, while processes or steps are presented in a given order, alternative embodiments may perform routines having steps in a different order, and some processes or steps may be deleted, moved, added, subdivided, combined, and/or modified to provide alternative or sub-combinations. Each of these processes or steps may be implemented in a variety of different ways. Also, while processes or steps are at times shown as being performed in series, these processes or steps may instead be performed in parallel, or may be performed at different times.

I claim:

1. A non-invasive wound closure device, comprising:
two elongated side strips, each of the side strips having a bandage adhered to a lower surface, an outer surface of the bandage having an adhesive thereon,
each side strip has an elongated protrusion on an upper surface of the side strip that extends longitudinally along the side strip, the protrusion having a top face, a perpendicular face on one side thereof, and an angled undercut face forming a wedge tongue on an opposing side,
the perpendicular face having a plurality of holes that pass through the protrusion, and
a securing cord that is threaded alternately through the holes of a first one of the side strips and a second one of the side strips; such that
the side strips are placed on either side of an open wound, a user of the device pushing the side strips into a desired position on the sides of the wound, the adhesive of the bandage adhering to undamaged skin around the wound,
the side strips then being secured in position by the cord, the cord being tied off to hold the side strips in position, and
if the user needs to reopen the wound, the cord is loosened and the side strips are pulled away from each other, the side strips being restored to their secured position to again close the wound.

2. The non-invasive wound closure device of claim 1, wherein:
the angled undercut face of the protrusion having an elongated groove for receiving the cord, and
the angled undercut face further having a plurality of protuberances.

3. The non-invasive wound closure device of claim 1, wherein:
a rigid slider with a groove therein, the groove conforming in shape to paired protrusions of the two side strips, the slider being installed on the paired protrusions to further secure the two side strips in position.

4. The non-invasive wound closure device of claim 3, wherein:
the rigid slider comprises a plurality of discrete slider sections, such that the slider can conform to a curved wound.

5. The non-invasive wound closure device of claim 3, wherein:
the rigid slider on the sides has a hole matching the protuberances, forming together with the hole a locking mechanism for the rigid slider.
